(19) United States
(12) Patent Application Publication
(10) Pub. No.: US 2003/0138906 A1
(43) Pub. Date: Jul. 24, 2003

(54) Fluorescence test for measuring heterotrophic bacteria in water

(76) Inventors: Ingun Tryland, Oslo (NO); Leonila Fijardo Hermansen, Haslum (NO)

Correspondence Address: DUNLAP, CODDING & ROGERS P.C., PO BOX 16370, OKLAHOMA CITY, OK, 73114, US

(21) Appl. No.: 10/280,870

(22) Filed: Oct. 24, 2002

Related U.S. Application Data
(60) Provisional application No. 60/337,360, filed on Nov. 5, 2001.

(51) Int. Cl. C12P 21/06

(52) U.S. Cl. 435/69.1

(57) ABSTRACT

A rapid method for measuring heterotrophic bacteria (or total viable organisms—TVO) in samples of drinking water. The water samples are diluted to reduce the masking effect of fast-growing bacteria over slow-growing bacteria. Subsamples are mixed with a growth medium, incubated, and analyzed for fluorescence whereby a semi-quantitative or pass-fail determination of heterotrophic bacteria in the water sample can be made.

### CROSS-REFERENCE TO RELATED APPLICATIONS

The present application claims benefit of U.S. Provisional application No. 60/337,360, filed Nov. 5, 2001.

### BACKGROUND

The present invention relates to a rapid method for measuring heterotrophic bacteria, or total viable organisms, in a water sample, and more particularly, in a drinking water sample.

The heterotrophic plate count (HPC) is a useful general indicator of the microbial quality of drinking water, particularly for monitoring the effects of water treatment processes and for monitoring the water quality during distribution. However, the analysis times for standard HPC methods are long (2-7 days) and generally do not allow technicians to take corrective actions on waters having substandard quality. Therefore, there is a need for more rapid methods for effectively monitoring the numbers of heterotrophic bacteria (also referred to as Total Viable Organisms or TVO) in drinking water.

### DETAILED DESCRIPTION OF THE INVENTION

The heterotrophic plate count is a method used to detect colony forming units (cfu) of aerobic and faculative anaerobic heterotrophic bacteria in water. This diverse group of bacteria consists of different species and strains that require very different optimal conditions for forming colonies on agar. Under certain sets of conditions, some strains will grow fast, and others slow. This is also the case when heterotrophic bacteria are grown in liquid media. Using automated fluorescence methods (e.g., COLIFAST CA), the fluorescence detection of some bacterial species is likely to require longer incubation times than others. These differences can be caused by bacteria having longer lag phases, longer generation times, or lower enzyme activities per cell. This can be caused by genetic differences between strains, but also by the physiological condition of the cells in a sample. For example, the physiological state of a cell can be influenced by conditions such as nutrition level, temperature or residual chlorine in the water sample. Chlorine has been shown to extend the lag period of the cells causing the cells to require longer incubation times before detection can occur, compared to unchlorinated water with the same composition of strains.

In the context of the method contemplated herein, bacteria that need longer incubation times (e.g. longer than about 20-24 hours under preferred conditions) to develop detectable fluorescence for any reason, are referred to as “slow growers” or “slow-growing bacteria”. Bacteria that require a shorter incubation time (e.g. less than about 18 hours under preferred conditions) and thus which can be detected relatively faster are referred to as “fast growers” or “fast-growing bacteria”.

The present invention contemplates a detection system in which enzyme activity of heterotrophic bacteria is used to effect a measurable indication of drinking water quality. The objective of the present invention is to provide a rapid tool for water plant operators and other users to both simplify and reduce the time-to-result of traditional HPC tests.

A typical range of heterotrophic bacteria found in drinking water includes species of Pseudomonas, Flavobacterium, Aeromonas, Acinetobacter, Alkaligens, Micorcoccus and Bacillus.

More particularly, the following bacteria are among the heterotrophic bacteria that can be measured by the present test: Pseudomonas aeruginosa, Flavobacterium breve, Acinetobacter lwoffi, Bacillus licheniformis, Pseudomonas fluorescens, Pseudomonas fragi, Enterobacter aerogenes, Enterococcus faecalis, Staphylococcus aureus, Citrobacter freundii, Klebsiella oxytoca, Serratia marcescens, Escherichia coli, and Hafnia alvei for example.

Development of a semi-quantitative method for heterotrophic bacteria in drinking water is complicated by several conditions, for example, the heterogeneity of the bacterial population (e. g. “slow-growers” versus “fast-growers”, i.e., bacteria with high enzyme production versus low enzyme production) and the impact of chlorine or disinfectant stress on bacteria, causing reduced bacterial lag phase, growth rate and enzyme activity. The length of the lag-phase for instance depends on how stressed the bacteria are. The growth rate (generation time) may vary widely among the different bacteria (10 min to several hours or days), but the generation time also depends on available nutrients, temperature, pH and other environmental factors. A particular bacterial strain could therefore be defined as a “slow-grower” under some conditions, but defined as a “fast-grower” under other conditions which occur at different times at the same site.

For example, in one test, Pseudomonas aeruginosa and Flavobacterium breve were found to be slow-growers while Acinetobacter lwoffi, Bacillus licheniformis, Pseudomonas fluorescens, Pseudomonas fragi, Enterobacter aerogenes, Enterococcus faecalis, Staphylococcus aureus, Citrobacter freundii, Klebsiella oxytoca, Serratia marcescens, Escherichia coli and Hafnia alvei were found to be fast-growers (when analysed from pure culture at 37° C.).

Using a liquid medium, low numbers of fast-growing bacteria will totally mask the fluorescent signal from slow-growing bacteria, independent of the number of slow-growing bacteria, hence giving poor correlation between the reference plate count and the time to detect (TTD) a fluorescent signal. For example, this problem is important when analysing water from the Norwegian drinking water distribution system where the chlorine doses are low and fast-growing bacteria are randomly distributed in the water samples (in general at low levels (0-5 cfu/ml)). If a fast-growing bacterium is present in a water sample, the TTD could be about 8 hours while another water sample, without fast-growing bacteria, but containing in excess of 100 slow-growing bacteria, may show a TTD exceeding 20 h. Even subsamples from the same water sample may show a variation in TTD of from 8-18 h, dependent on the occasional presence of one or several fast-growing bacteria in one or more of the subsamples thereby masking the possible presence of slow-growing bacteria into the subsample.

In the present invention, the water sample is diluted in a predetermined manner so as to minimize the number of fast-growing organisms in the subsamples tested without excessively diluting the numbers of slow-growing organisms. Otherwise, fluorescence from the fast-growers might “swamp” or “mask” the fluorescence from the slow-growers, thus inhibiting detection of the latter, even if the slow-growers are present in much larger numbers than fast-growers.

It is acceptable to suffer the loss of inclusion of a small number of fast-growing bacteria in an effort to detect a much greater number of slow-growing bacteria because a primary value of the TVO or heterotrophic bacterial detection test is as a monitor of general water quality rather than as a test for a specific target microorganism. Furthermore, fast-growing bacteria are generally present in relatively low numbers in finished drinking water and therefore their presence is generally not as important as slow-growers, as a practical matter.

The test method contemplated herein is composed of several basic steps, including:

(1) diluting the original water sample (according to a predetermined dilution protocol), thereby reducing the chance for fast-growing bacteria to be present in the tested water sample,

(2) taking several subsamples from each diluted sample, mixing each with a growth medium such as a TVO liquid medium and incubating the subsamples,

(3) running the test for a sufficient time so that most of the slow-growing bacteria will be detected in each incubated subsample if present, and

(4) determining the number of positive vials (after a given incubation time) and transforming this to a semi-quantitative bacterial number or a pass-fail result based on data previously collected at the same site or test location (empirical semi-quantification).

Preferably, a dilution protocol is developed which is specific for each test location, for example in the manner shown below, thereby developing a baseline dilution procedure for that test location. To develop a dilution protocol, the steps listed below can be followed. This procedure is but one example of a procedure for determining a dilution protocol and it is not intended that the present invention necessarily be limited to using procedures for determining a dilution protocol such as shown herein.

(1) An initial water sample is diluted with sterile water using an initial dilution factor, for example 1:10. Portions of the diluted water sample are mixed with a TVO growth medium such as described elsewhere herein. This is done four times for example to create four subsamples for further incubation.

(2) The four subsamples are incubated at a predetermined temperature (e.g. 28-32° C.) for a predetermined period such as 16 hours (or from 10-18 hours). The initial incubation time should be long enough to activate most fast-growers, but short enough not to activate most slow-growers as explained below.

(3) After initial incubation, a fluorescence measurement is determined for each subsamples (via a standard excitation/emission procedure). If the fluorescence exceeds a predetermined threshold (e.g., 100 ppb of methylumbelliferone (MU)), the subsample is designated as positive for fast-growers. If the threshold isn't exceeded, the subsample is designated as negative for fast-growers.

(4) Incubation of the negative subsample is continued for a total of 30 hours (or, for example, from about 24 up to about 36 hours). If the fluorescence measurement exceeds the predetermined threshold after the continued incubation period, the subsample is designated as positive for slow-growers. In an alternative embodiment, incubation of the subsample can be continued for from 36-46 hours, if at that specific test site it is desired to extend the test further.

(5) A subsample is considered to be positive for fast-growers if the fluorescence measurement exceeds the predetermined threshold (e.g., 100 ppb MU) within a “short” incubation time for detecting fast-growers, e.g. less than about 18 hours, at 30° C., preferably 10-16 hours.

(6) A subsample is considered to be positive for slow-growers if (a) the fluorescence measurement exceeds the predetermined threshold (e.g., 100 ppb MU) within a “long” incubation time for detecting slow-growers, e.g., 26-34 hours at 30° C., or preferably 30 hours, and (b) the subsample is not positive for fast-growers.

(7) The above process is repeated with several different initial dilution factors (e.g., 1:10, 1:20, 1:50, 1:100) to obtain results with several different initial dilutions.

(8) An optimal dilution factor is then selected from the various initial dilution factors tested which routinely results in no more than one subsample being positive for fast-growers (e.g., out of four subsamples) but which routinely results in at least one subsample (e.g., out of four) which is positive for slow-growers after the total incubation period. This selected dilution factor is used in future testing at the particular testing location unless conditions change significantly.

### EXAMPLE

### Analysis of Drinking Water from the Norwegian Distribution System

Water samples were diluted using a selected dilution factor of 1:100 (1 ml water sample and 99 ml dilution water), 9 ml subsamples of the diluted samples were mixed with 1 ml TVO-medium (see below) in vials. Four parallels (vials) were used as subsamples. Vials were incubated in a COLIFAST ANALYSER (CA) at 30° C. Sub-portions of each subsample were taken after 30 h and 42 h for fluorescence determination and the number of positive vials was determined (positive was defined as a fluorescence measurement equal to or greater than a 100 ppb MU threshold value).

An empirically based semi-quantification schedule was arrived at to estimate numbers of slow-growing bacteria in the original water sample:

0 or 1 positive vials: <25 cfu/ml

2 positive vials: 20-70 cfu/ml

3 positive vials: 50-100 cfu/ml

4 positive vials: >100 cfu/ml

A “pass/fail” schedule was determined to make a decision about water quality based on “failure” if the original water sample equal or exceeded 100 cfu/ml.:

0-3 positive vials : <100 cfu/ml (i.e., “pass”).

4 positive vials: ≧100 cfu/ml (i.e., “fail”).

(Note: To derive a location-specific semi-quantification table, a large number of samples are analyzed with the selected dilution factor. The results are compared to a conventional estimation method. A semi-quantification table is chosen based on the resulting correlations between the results obtained using the dilution protocol described herein and the results obtained from the standard method.)

The results obtained after 30 h by the CA at 30° C. were then compared to results obtained with the standard Heterotrophic plate count (HPC) method on reference agar (3 days (68-72 h) at 22° C.). In one experiment, 34 samples of drinking water, and drinking water contaminated with river water, were tested. The present semi-quantification method when compared with reference method gave ≧88% agreement. The present pass-fail method when compared with reference method gave ≧88% agreement. In another experiment, 96 samples of drinking water, and drinking water contaminated with river water, were tested. The present semi-quantification method when compared with reference method gave ≧83% agreement. The present pass-fail method when compared with reference method gave ≧85% agreement.

The results obtained after 42 h by the CA (30° C.) were then compared to results obtained with the standard HPC method on reference agar after 5 days at 22° C. Thirty-four samples of drinking water, and drinking water contaminated with river water, were tested. The present semi-quantification method when compared with the HPC reference method gave ≧79% agreement. The present pass-fail method when compared with the HPC reference method gave ≧97% agreement.

Growth Medium and Methods

Optimised TVO-Medium and Procedure for Preparing the Medium:

1. Solution A: Dissolve 2.83 g TVO-basic-medium* (1 bottle) in 90 ml distilled water in a Duran bottle. Stir to dissolve. Autoclave at 121° C. for 15 min to sterilize. Allow cooling to room temperature.

2. Solution B: Add 5 ml acetone and 5 ml Dimethyl sulfoxide (DMSO) successively to one 115 mg-bottle of TVO-substrate-mix**. Mix well to dissolve.

3. Final TVO-medium: Add Solution B (10 ml) to Solution A (90 ml) by sterile filtration (0.2 μm pore size)***. Stir for at least 10 min.

Precipitation may be observed in the final TVO-medium, and it is therefore recommended to fill media in vials immediately. Stir well to obtain a “homogeneous” solution and pipette 1 ml final TVO-medium to pre-autoclaved vials (95-100 vials) using sterile technique. Vials containing final media may be stored in the dark at 4-8° C. for four weeks. Precipitation may be seen in the vials, but this will not reduce the performance of the test.

*TVO-basic medium: Bacto Yeast Extract (0.5 g), Bacto Proteose Peptone (0.5 g), Casamino Acids (0.5 g), Dextrose (0.5 g), Soluble Starch (0.2 g), Sodium Pyruvate (0.3 g), Potassium Phosphate(3H₂O) (0.28 g), Magnesium Phosphate (0.05 g), distilled water (90 ml). This medium is 10× concentrated R2A medium without agar, with the additional modifications that Soluble Starch concentration is reduced from 0.5 g to 0.2 g (to reduce precipitation of starch in the liquid medium), and that Potassium Phosphate (3H₂O) is reduced from 0.3 g to 0.28 g (to obtain pH 7.0).

**TVO substrate mix: 4-methylumbelliferyl-(-D-glucoside (50 mg), 4-methylumbelliferyl-phosphate (50 mg), 4-methylumbelliferyl-palmitate (15 mg).

***The substrate solution or the final medium must not be autoclaved because the substrates will auto-hydrolyse at high temperatures. The substrate solution is therefore added to the basic medium using sterile filtration techniques.

Preparation of Subsamples:

Add 9 ml water samples (or dilutions of water samples) to vials containing 1 ml final TVO-medium.

Fluorescence Measurement:

In one embodiment of the present invention, a laboratory fluorometer is used to measure fluorescence. In this embodiment, a fluorometer such as a TURNER DESIGNS TD-700 fluorometer can be used. The TD-700 has two optical filters: an excitation filter with a 380 nm narrow band pass to provide a wavelength of 380 nm for exciting the MU, and an emission filter with a 450 nm narrow band pass to detect an emission wavelength of 450 nm from the MU. The TD-700 is single point calibrated using pure TVO media for setting the optimal sensitivity and range for the fluorometer. Fluorescence measurements in this embodiment preferably are taken after the predetermined inculation period and preferably are incubated at 28°-32° C. In another embodiment, as described elsewhere herein, fluorescence is automatically measured using a COLIFAST CA.

The present invention is not to be limited in scope by the specific embodiments described herein, since such embodiments are intended as but single illustrations of one aspect of the invention and any functionally equivalent embodiments are within the scope of this invention. Indeed, various modifications of the invention is addition to those shown and described herein will become apparent to those skilled in the are from the foregoing description. Such modifications are intended to fall within the scope of the appended claims.

What is claimed is:

1. A method of detecting and measuring heterotrophic bacteria in a water sample, comprising:
providing a water sample to be tested;
diluting the water sample according to a predetermined dilution protocol thereby forming a diluted water sample, wherein the predetermined dilution protocol dilutes the water sample so as to minimize the numbers of fast-growing bacteria in a plurality of subsamples derived therefrom such that the presence of slow-growing bacteria in the subsamples is not masked by the presence of fast-growing bacteria in the subsamples;
mixing each subsample with a quantity of a growth medium comprising a fluorogenic substrate;
incubating the subsamples for a predetermined incubation period sufficient to enable slow-growing bacteria present in the subsample to act on the fluorogenic substrate to produce a fluorescent product;
obtaining a fluorescence measurement from each subsample after the predetermined incubation period, wherein the subsample is designated as positive when the fluorescence measurement equals or exceeds a predetermined threshold concentration of the fluorescent product and the subsample is designated as negative when the fluorescence measurement is less than the predetermined threshold concentration of the fluorescent product; and
making a semi-quantitative estimate of the number of heterotrophic bacteria in the water sample based on the number of subsamples designated as positive.

2. The method of claim 1 wherein in the step of incubating the subsamples, the predetermined incubation period is from about 24 to about 36 hours.

3. The method of claim 1 wherein in the step of incubating the subsamples, the predetermined incubation period is from about 26 to about 32 hours.

4. The method of claim 1 wherein in the step of incubating the subsamples, the predetermined incubation period is from about 34 to about 46 hours.

5. The method of claim 1 wherein in the step of incubating the subsamples, the predetermined incubation period is from about 36 to about 42 hours.

6. The method of claim 1 wherein in the step of mixing each subsample with a quantity of a growth medium, the fluorogenic substrate comprises three fluorogenic compounds.

7. The method of claim 1 wherein in the step of incubating the subsamples, the fluorescent product is methylumbelliferone.

8. The method of claim 1 wherein in the step of providing the subsamples, four subsamples are provided.

9. The method of claim 1 wherein in the step of incubating the subsamples, the subsamples are incubated at a temperature of between about 20° C. and about 37° C.

10. The method of claim 1 wherein in the step of incubating the subsamples, the subsamples are incubated at a temperature of between about 28° C. and about 37° C.

11. The method of claim 1 wherein in the step of incubating the subsamples, the subsamples are incubated at a temperature of between about 28° C. and about 32° C.

12. The method of claim 1 wherein in the step of making a semi-quantitative estimate, when all subsamples are designated as positive, the water sample is concluded as having at least 100 CFU/ml.

13. The method of claim 1 wherein in the step of making a semi-quantitative estimate, when at least one subsample is designated as negative, the water sample is concluded as having less than 100 CFU/ml.

14. The method of claim 8, wherein in the step of making a semi-quantitative estimate, the water sample is concluded as having at least 100 CFU/ml when all four samples are designated as positive.

15. The method of claim 8, wherein in the step of making a semi-quantitative estimate, the water sample is concluded as having less than 100 CFU/ml when at least one of the four subsamples is designated as negative.

16. The method of claim 1 wherein in the step of diluting the water sample, the predetermined dilution protocol is altered to increase the dilution of the water sample when more than one of the subsamples is determined to be positive for fast-growing bacteria.

17. The method of claim 1 wherein the step of obtaining a fluorescence measurement is performed manually or automatically.
